(19) United States
(12) Patent Application Publication
(10) Pub. No.: US 2026/0306206 A1
(43) Pub. Date: Oct. 8, 2026

(54) PROTECTION SLEEVE FOR REUSABLE HANDHELD INSTRUMENT

(71) Applicants: STRYKER CORPORATION, Portage, MI (US); STRYKER EUROPEAN OPERATIONS LIMITED, Co. Cork (IE)

(72) Inventors: Josh Lazar, Fremont, CA (US); Danny Lai, Portage, MI (US); Tra Ngo, Portage, MI (US); Brandon Cohen, Portage, MI (US)

(73) Assignees: STRYKER CORPORATION, Portage, MI (US); STRYKER EUROPEAN OPERATIONS LIMITED, Co. Cork (IE)

(21) Appl. No.: 19/451,566

(22) Filed: Jan. 16, 2026

Related U.S. Application Data
(63) Continuation of application No. PCT/US2024/041595, filed on Aug. 9, 2024.
(60) Provisional application No. 63/519,987, filed on Aug. 16, 2023.

(51) Int. Cl. A61B 46/10; A61B 17/12; A61B 17/34; A61B 17/00; A61M 25/00

(52) U.S. Cl. CPC A61B 46/10; A61B 17/1214; A61B 17/3498; A61B 2017/0046; A61B 2017/00907; A61B 2017/12063; A61M 25/0014

(57) ABSTRACT

A sterile adapter for electrically coupling an electrically conductive wire of a medical device to an electrical port of a handheld instrument. The sterile adapter comprises a sterile sleeve sized to removably contain the handheld instrument, a hub assembly affixed to the sterile sleeve for receiving a proximal end of the electrically conductive wire of the medical device, and an electrically conductive shaft affixed to the hub assembly and contained within the sterile sleeve. The electrically conductive shaft has a distal end affixed to the hub assembly and a proximal end configured for being inserted into the electrical port of the handheld instrument. The electrically conductive shaft has a lumen into which the proximal end of the electrically conductive wire of the medical device may be slidably disposed when received by the hub assembly.

### RELATED APPLICATION DATA

The application is a continuation of International Patent Application No. PCT/US2024/041595, filed on August 9, 2024, which claims the benefit of U.S. Provisional Patent Application Serial No. 63/519,987, filed on August 16, 2023, the entire disclosures of all of which are hereby incorporated herein by reference in their entirety into the present application.

### FIELD OF THE INVENTION

The present disclosure relates generally to medical devices, and more particularly, to a sterile barrier for isolating a reusable hand-held instrument from a sterile medical device.

### BACKGROUND

During medical procedures that require introduction of a sterile medical device through an opening (e.g., a minimally invasive opening or a surgical opening) within a patient, it is desirable to maintain a sterile field around the opening into which the medical device is to be introduced. For example, in a typical medical procedure, a sterile barrier (e.g., a drape) is placed over the patient to establish a sterile field for introduction of the medical device into the patient. However, there is often a need to breach the sterile barrier to make an electrical connection between the sterile medical device and a handheld instrument for conveying electrical energy or electrical communication to and/or from the sterile medical device without compromising the sterility of the sterile field.

For example, in one medical procedure, vaso-occlusive devices (e.g., vaso-occlusive coils, such as those described in U.S. Patent No. 4,994,069, which is expressly incorporated herein by reference) may be endovascularly introduced into a patient and delivered into an aneurysmal sac until the aneurysmal sac is completely filled with vaso-occlusive devices. One highly desirable means of delivering vaso-occlusive coils into an aneurysmal sac employs an electrolytic detachment procedure, such as that described in U.S. Patent No. 5,122,136, which is expressly incorporated herein by reference. After loading an electrically conductive delivery wire (e.g., composed of stainless steel) with an attached vaso-occlusive coil within a delivery catheter previously intravascularly introduced into the patient and distally advancing the delivery wire to insert the vaso-occlusive coil into the aneurysmal sac, such electrolytic detachment procedure involves severing the vaso-occlusive coil from the distal end of delivery wire by the application of a small electric current through the delivery wire to an electrolytically severable joint between the vaso-occlusive coil and the distal end of the delivery wire, thereby permanently delivering the detached vaso-occlusive coil into the aneurysmal sac.

A handheld electrolytic detachment device, such as the InZone® Detachment System, manufactured by Stryker® Neurovascular, may be employed to deliver the small electrical current to the electrolytically severable joint between the vaso-occlusive coil and the distal end of the delivery wire, and detect and report detachment of the vaso-occlusive coil from the delivery wire. One embodiment of such a handheld electrolytic detachment device includes a wire insertion port into which the proximal end of the delivery wire may be inserted for delivery of the electrical current to the electrolytically severable joint in either a monopolar arrangement or a bipolar arrangement. In the monopolar arrangement, the handheld electrolytic detachment device includes a power terminal to which the proximal end of the delivery wire is coupled, and a ground terminal to which a ground electrode placed in contact with the patient (e.g., via percutaneous insertion into the patient) is coupled via an electrical cable (e.g., via insertion of a connector of the electrical cable into a ground port), whereas, in the bipolar arrangement, the handheld electrolytic detachment device includes a power terminal and a ground terminal to which the proximal end of the delivery wire is coupled. The handheld electrolytic detachment device may be operated (e.g., by depressing a button) on handle to perform one or more electrolytic detachment cycles (i.e., deliver one or more cycles of electrical current) to the electrolytically severable joint between the vaso-occlusive coil and the distal end of the delivery wire.

Handheld instruments (such as handheld electrolytic detachment devices) that are proximally connected to sterile medical devices for introduction into a patient may either be designed to be disposable (single-use) or reusable. Single-use handheld instruments, of course, add a significantly high fixed cost to each medical procedure and are disposed of after each use, contributing to global waste production, and are, therefore, not optimal. In contrast, reusable handheld instruments must be sterilized before each use, and thus, must be made very durable to withstand repeated sterilizing cycles, adding considerable manufacturing costs. Furthermore, thorough and proper sterilization of a reusable handheld instrument requires particular care and is consequently a time-consuming operation. Also, adequate sterilization of reusable handheld instruments cannot be assured, especially if such reusable handheld instruments have working channels or other such difficult-to-clean portions, such as the wire insertion port of a handheld electrolytic detachment device.

To eliminate the need to sterilize reusable instruments, some manufacturers provide inexpensive sterile sleeves that completely cover the reusable instruments, thereby isolating the reusable instruments from the associated sterile medical devices that are introduced into the patient. In these cases, the need to sterilize such reusable instruments is eliminated. However, isolating reusable instruments from sterile medical devices is more complicated when such reusable instruments directly interface with a sterile medical device that requires an electrical connection through the sterile sleeve. Additional complications may occur when reusable handheld instruments, such as handheld electrolytic detachment devices, have one or more controls that need to be accessed by a medical practitioner.

There, thus, remains a need to provide an effective means for isolating a reusable handheld instrument from a sterile medical device that requires an electrical connection therebetween.

### SUMMARY

In accordance with one aspect of the present invention, a sterile adapter for electrically coupling an electrically conductive wire of a medical device to an electrical port of a handheld instrument is provided.

The sterile adapter comprises a sterile sleeve sized to removably contain the handheld instrument. In one embodiment, the sterile sleeve is optically transparent. In another embodiment, the sterile sleeve has a sealable opening into which the handheld instrument may be inserted, and from which the handheld instrument may be removed. In an optional embodiment, the sterile adapter further comprises a clamping mechanism affixed to the sterile sleeve. The clamping mechanism is configured for grasping the handheld instrument when contained within the sterile sleeve.

The sterile adapter further comprises a hub assembly affixed to the sterile sleeve for receiving a proximal end of the electrically conductive wire of the medical device. In one embodiment, the hub assembly comprises a hub affixed to the sterile sleeve, and a rotating hemostasis valve (RHV) to which the hub is affixed, for reversibly affixing the electrically conductive wire of the medical device relative to the hub.

The sterile adapter further comprises an electrically conductive shaft (e.g., a hypotube) affixed to the hub assembly and contained within the sterile sleeve. The electrically conductive shaft has a distal end affixed to the hub assembly and a proximal end configured for being inserted into the electrical port of the handheld instrument. The electrically conductive shaft has a lumen into which the proximal end of the electrically conductive wire of the medical device may be slidably disposed when received by the hub assembly. In one embodiment, the lumen of the electrically conductive shaft is closed at the proximal end of the electrically conductive shaft, and the proximal end of the electrically conductive shaft has an electrically conductive epoxy cap. In another embodiment, the electrically conductive shaft has a proximal shaft portion, a distal shaft portion, and an electrically insulative element disposed between the proximal and distal shaft portions. In this embodiment, the proximal shaft portion may be a proximal hypotube, the second shaft portion may be a distal hypotube, one of the proximal and distal hypotubes may be partially inserted into the other of the proximal and distal hypotubes to create an overlapping region, and the electrically insulative element may be an electrically insulative layer disposed between, and spanning the overlapping region of the proximal and distal hypotubes.

In accordance with a second aspect of the present inventions, a medical kit comprises a medical device comprising an electrically conductive wire, and a handheld instrument comprising an electrical port having at least one electrical terminal. In one embodiment, the medical device is a vaso-occlusive assembly that comprises a vaso-occlusive device electrolytically attached to a distal end of the electrically conductive wire, and the handheld instrument is a handheld electrolytic detachment device.

The medical kit further comprises a sterile adapter comprising a sterile sleeve sized to removably contain the handheld instrument. In one embodiment, the handheld instrument may comprise at least one control for delivering electrical current to the medical device via the electrical terminal(s), in which case, the sterile sleeve may be optically transparent. In another embodiment, the sterile sleeve has a sealable opening into which the handheld instrument may be inserted, and from which the handheld instrument may be removed. In an optional embodiment, the sterile adapter further comprises a clamping mechanism affixed to the sterile sleeve. The clamping mechanism is configured for grasping the handheld instrument when contained within the sterile sleeve.

The sterile adapter further comprises a hub assembly affixed to the sterile sleeve for receiving a proximal end of the electrically conductive wire of the medical device. In one embodiment, the hub assembly comprises a hub affixed to the sterile sleeve, and an RHV to which the hub is affixed, for reversibly affixing the electrically conductive wire of the medical device relative to the hub.

The sterile adapter further comprises an electrically conductive shaft (e.g., a hypotube) affixed to the hub assembly and contained within the sterile sleeve. The electrically conductive shaft has a distal end affixed to the hub assembly and a proximal end configured for being inserted into the electrical port of the handheld instrument, such that the electrical terminal(s) of the handheld instrument electrically contacts the electrically conductive shaft. The electrically conductive shaft has a lumen into which the proximal end of the electrically conductive wire of the medical device may be slidably disposed when received by the hub assembly, such that electrically conductive wire is electrically coupled to the electrical terminal(s) of the handheld instrument via the electrically conductive shaft of the sterile adapter. In one embodiment, the lumen of the electrically conductive shaft is closed at the proximal end of the electrically conductive shaft, and the proximal end of the electrically conductive shaft has an electrically conductive epoxy cap. In another embodiment, the electrically conductive shaft has a proximal shaft portion, a distal shaft portion, and an electrically insulative element disposed between the proximal and distal shaft portions. In this embodiment, the proximal shaft portion is a proximal hypotube, the second shaft portion is a distal hypotube, one of the proximal and distal hypotubes is partially inserted into the other of the proximal and distal hypotubes to create an overlapping region, and the electrically insulative element is an electrically insulative layer disposed between, and spanning the overlapping region of the proximal and distal hypotubes.

In one embodiment, the electrical terminal(s) of the handheld instrument comprises a power terminal disposed in the electrical port, the medical device comprises a power terminal disposed on the electrically conductive wire, and the sterile adapter comprises a power terminal disposed on the electrically conductive shaft, such that power terminal of the medical device electrically contacts the power terminal of the sterile adapter when the proximal end of the electrically conductive wire is inserted into the lumen of the electrically conductive shaft of sterile adapter, and such that the power terminal of the handheld instrument electrically contacts the power terminal of the sterile adapter when the proximal end of the electrically conductive shaft of the sterile adapter is inserted into the electrical port of the handheld instrument. In this embodiment, the electrical terminal(s) of the handheld instrument may further comprise a ground terminal, the medical device may further comprises a ground terminal disposed on the electrically conductive wire, and the sterile adapter may further comprise a ground terminal disposed on the electrically conductive shaft, such that ground terminal of the medical device electrically contacts the ground terminal of the sterile adapter when the proximal end of the electrically conductive wire is inserted into the lumen of the electrically conductive shaft of sterile adapter, and such that the ground terminal of the handheld instrument electrically contacts the ground terminal of the sterile adapter when the proximal end of the electrically conductive shaft of the sterile adapter is inserted into the electrical port of the handheld instrument.

In this embodiment, the electrically conductive wire of the vaso-occlusive assembly may comprise a first proximal shaft portion, a first distal shaft portion, and a first electrically insulative element disposed between the first proximal shaft portion and the first distal shaft portion, thereby forming the power terminal and the ground terminal of the vaso-occlusive assembly, and the electrically conductive shaft of the sterile adapter may comprise a second proximal shaft portion, a second distal shaft portion, and a second electrically insulative element disposed between the second proximal shaft portion and the second distal shaft portions, thereby forming the power terminal and the ground terminal of the sterile adapter. The first proximal shaft portion may be a first proximal hypotube, the first distal shaft portion ma be a first distal hypotube, one of the first proximal hypotube and the first distal hypotube may be partially inserted into the other of the first proximal hypotube and the first distal hypotube to create an overlapping region, the first electrically insulative element may be a first electrically insulative layer disposed between, and spanning the overlapping region, of the first proximal hypotube and the first distal hypotube, and the second proximal shaft portion may be a second proximal hypotube, the second distal shaft portion may be a second distal hypotube, one of the second proximal hypotube and the second distal hypotube may be partially inserted into the other of the second proximal hypotube and the second distal hypotube to create an overlapping region, and the second electrically insulative element may be a second electrically insulative layer disposed between, and spanning the overlapping region, of the second proximal hypotube and the second distal hypotube.

Other and further aspects and features of embodiments will become apparent from the ensuing detailed description in view of the accompanying figures.

### BRIEF DESCRIPTION OF THE DRAWINGS

The drawings illustrate the design and utility of preferred embodiments of the disclosed inventions, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. Further, an aspect or an advantage described in conjunction with a particular embodiment of the disclosed inventions is not necessarily limited to that embodiment and can be practiced in any other embodiments even if not so illustrated.

In order to better appreciate how the above-recited and other advantages and objects of the disclosed inventions are obtained, a more particular description of the disclosed inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a vaso-occlusive treatment system constructed in accordance with one embodiment of the disclosed inventions, particularly showing a vaso-occlusive device in a delivery configuration;

FIG. 2 is a plan view of the vaso-occlusive treatment system of FIG. 1, particularly showing the vaso-occlusive device in a deployed configuration;

FIG. 3 is a close-up, partially-cutaway, cross-sectional view of a portion of a vaso-occlusive assembly of the vaso-occlusive treatment system of FIG. 1;

FIG. 4 is a top view of a hand-held electrolytic detachment device of the vaso-occlusive treatment system of FIG. 1;

FIG. 5 is a top view of a sterile adapter of the vaso-occlusive treatment system of FIG. 1;

FIG. 6A is a front view of a sterile sleeve of the sterile adapter of FIG. 5, particularly showing the opening of the sterile sleeve while in an open state;

FIG. 6B is a front view of a sterile sleeve of the sterile adapter of FIG. 5, particularly showing the opening of the sterile sleeve while in a closed and sealed state;

FIG. 7 is a cross-sectional view of proximal end of a bipolar core wire of a vaso-occlusive assembly of the vaso-occlusive treatment system of FIG. 1 inserted within a bipolar electrically conductive sleeve of the sterile adapter of FIG. 5;

FIG. 8 is a cross-sectional view of proximal end of a monopolar core wire of a vaso-occlusive assembly of the vaso-occlusive treatment system of FIG. 1 inserted within a monopolar electrically conductive sleeve of the sterile adapter of FIG. 5;

FIG. 9 is one embodiment of the sterile adapter of FIG. 5, particularly showing an optional clamping mechanism for the electrolytic detachment device of FIG. 4; and

FIG. 10 is another embodiment of the sterile adapter of FIG. 5, particularly showing another optional clamping mechanism for the electrolytic detachment device of FIG. 4.

### DETAILED DESCRIPTION OF THE ILLUSTRATED EMBODIMENTS

The present disclosure is directed to a sterile adapter capable of physically isolating a handheld instrument from a sterile medical device having an electrically conductive wire, while also providing for an electrical connection between the handheld instrument and the sterile medical device. By physically isolating the handheld instrument from a sterile environment, the handheld instrument, rather than disposing of it right after use, may be reused between medical procedures without risk of cross-contamination. The sterile adapter comprises a sterile sleeve sized to contain the handheld instrument therein, a hub assembly affixed to the sterile sleeve for receiving a proximal end of the electrically conductive wire of the sterile medical device, and an electrically conductive shaft contained within the sterile sleeve.

The electrically conductive shaft has a distal end affixed to the hub assembly, and a proximal end configured for being inserted into an electrical port of the handheld instrument when contained within the sterile sleeve, such that proximal end of the electrically conductive shaft electrically contacts a power terminal within the electrical port of the handheld instrument. The electrically conductive shaft of the sterile adapter has a lumen in which the proximal end of the electrically conductive wire may be slidably disposed after being received by the hub assembly, such that the electrically conductive wire of the medical device is in electrical contact with the electrically conductive shaft, and thus, electrically coupled to the power terminal of the handheld instrument via the electrically conductive shaft. In this manner, the sterile sleeve of the sterile adapter physically isolates the handheld instrument from the sterile medical device (which resides in the sterile environment), while the sterile sleeve of the sterile adapter allows medical personnel to operate one or more controls, as well as visualizing signals from any indicators, on the handheld instrument, without breaching the sterile field, and while the hub assembly and electrically conductive shaft of the sterile adapter facilitates electrical connection between the sterile medical device and the handheld instrument without breaching the sterile field.

The handheld instrument and medical device may be bipolar in nature, in which case, the electrical port of the handheld instrument may have both a power terminal and a ground terminal, the electrically conductive wire of the medical device may have a proximal wire portion, a distal wire portion, and an electrically insulative element disposed therebetween, while the electrically conductive shaft of the sterile adapter is designed to mimic the electrical properties of the bipolar electrically conductive wire, and thus, may have a proximal shaft portion, a distal shaft portion, and an electrically insulative element disposed therebetween. When the proximal end of the electrically conductive shaft of the sterile adapter is inserted into the electrical port of the handheld instrument, the proximal shaft portion of the electrically conductive shaft electrically contacts one of the power terminal and the ground terminal of the handheld instrument, while the distal shaft portion of the electrically conductive shaft electrically contacts the other of the power terminal and the ground terminal of the handheld instrument. When the proximal end of the electrically conductive wire of the sterile medical device is received within the hub assembly and slidably disposed within the lumen of the electrically conductive shaft of the sterile adapter, the proximal wire portion of the electrically conductive wire of the sterile medical device electrically contacts the proximal shaft portion of the electrically conductive shaft of the sterile adapter, and thus electrically coupled to the one of the power terminal and ground terminal of the handheld instrument via the proximal shaft portion of the electrically conductive shaft, while the distal wire portion of the electrically conductive wire of the sterile medical device electrically contacts the distal shaft portion of the electrically conductive shaft of the sterile adapter, and thus electrically coupled to the other of the power terminal and ground terminal of the handheld instrument via the distal shaft portion of the electrically conductive shaft.

Alternatively, the handheld instrument and medical device may be monopolar in nature, in which case, the ground terminal of the handheld instrument will be remote from the power terminal within the electrical port for electrical connection to a ground electrode that can be placed into a contact with the patient. In this case, the electrically conductive shaft of the sterile adapter may electrically contact the power terminal of the handheld instrument when the proximal end of the electrically conductive shaft is inserted into the electrical port of the handheld instrument, while the electrically conductive wire of the sterile medical device may be electrically coupled to the power terminal of the handheld instrument via the electrically conductive shaft of the sterile adapter when the proximal end of the electrically conductive wire is received within the hub assembly and slidably disposed within the lumen of the electrically conductive shaft.

In one embodiment, the hub assembly of the sterile adapter may include a hub that is affixed to the sterile sleeve of the sterile adapter, such that a portion of the hub is external to the sterile sleeve for receiving the proximal end of the electrically conductive wire of the sterile medical device, while a portion of the hub is internal to the sterile sleeve for affixation to the electrically conductive shaft contained within the sterile sleeve. The hub assembly may also include a rotating hemostasis valve (RHV) to which the electrically conductive wire of the sterile medical device may be reversibly affixed relative to the hub. In this manner, the movement of the proximal end of the electrically conductive wire within the electrically conductive shaft of the sterile adapter may be prevented, thereby ensuring that the electrically conductive wire remains in electrical contact with the electrically conductive shaft.

The sterile adapter may optionally include a clamping mechanism configured for grasping the handheld instrument when contained within the sterile sleeve. In this manner, the movement between the proximal end of the electrically conductive shaft of the sterile adapter and the electrical port of the handheld instrument may be prevented, thereby ensuring that that the electrically conductive shaft remains in electrical contact with the power terminal and/or ground terminal of the handheld instrument.

In the embodiments described herein, the sterile medical device is described as a vaso-occlusive assembly having a vaso-occlusive device electrolytically detached to the electrically conductive wire of the vaso-occlusive assembly, while the handheld instrument is described as a handheld electrolytic detachment device. It should be appreciated, however, that sterile medical device can be any medical device that can be introduced into a patient and has electrical functionality, and the handheld instrument can be any handheld instrument that transmits and/or receives electrical current to or from the sterile medical device.

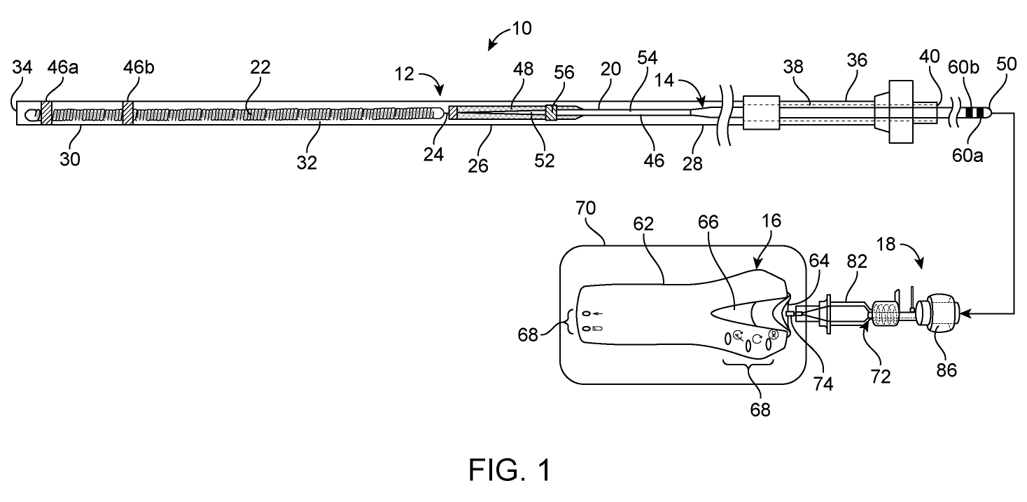

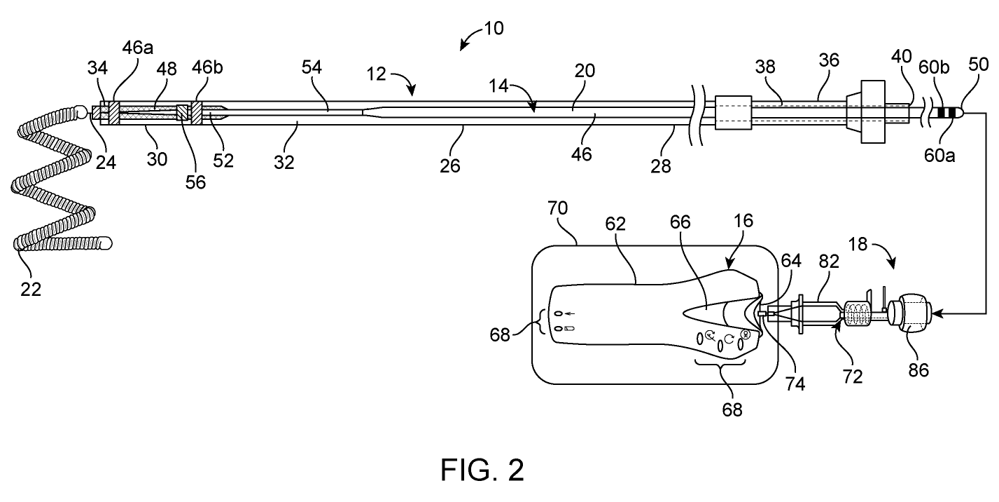

Referring to FIGS. 1-2, one embodiment of a vaso-occlusive treatment system 10 constructed in accordance with the disclosed inventions will now be described. The vaso-occlusive treatment system 10 comprises a delivery catheter 12, a vaso-occlusive assembly 14 slidably disposed within the delivery catheter 12, an electrolytic detachment device 16 to which the vaso-occlusive assembly 14 is removably affixed, and a sterile adapter 18 configured for physically isolating the electrolytic detachment device 16 from the vaso-occlusive assembly 14, while facilitating electrical connection between the electrolytic detachment device 16 and the vaso-occlusive assembly 14.

As will be discussed in further detail below, the vaso-occlusive assembly 14 comprises a delivery wire 20 and a vaso-occlusive device 22 detachable coupled to the delivery wire 20 via an electrolytically severable joint 24.

The delivery catheter 12 has a tubular configuration, and can, e.g., take the form of a micro-catheter, a sheath, or the like. The delivery catheter 12 comprises an elongate sheath body 26 having a proximal segment 28 and a distal segment 30, and an inner lumen 32 (shown partially in phantom) extending through the sheath body 26 between the proximal segment 28 and the distal segment 30, and in which the vaso-occlusive assembly 14 is housed. In alternative embodiments, the delivery catheter 12 may comprise two inner lumens (not shown) extending through the sheath body 26 between the proximal segment 28 and the distal segment 30, and in which two vaso-occlusive assemblies (not shown) may be respectively housed. The free end of the proximal segment 28 of the sheath body 26 remains outside of the patient and accessible to an operator (e.g., clinician or physician), while the remainder of the sheath body 26, including the distal segment 30, is sized and dimensioned to reach remote locations of the vasculature of the patient. The sheath body 26 has a suitable length for accessing a target tissue site within the patient from a vascular access point. The target tissue site depends on the medical procedure for which the delivery catheter 12 is used. For example, if the delivery catheter 12 is used to access vasculature in a brain of a patient from a femoral artery access point at the groin of the patient, the overall length of the sheath body 26 may be 125cm-200cm. The outer diameter of the sheath body 26 may be in the range of 3F-10F. In one embodiment, the outer diameter of the sheath body 26 may be uniform along the length of the sheath body 26. In another embodiment, the outer diameter of the sheath body 26 may taper in either a gradual fashion or a step-wise fashion from a first outer diameter of the proximal segment 28 to a second outer diameter at the distal segment 30 to facilitate navigation in tortuous vasculature. Although depicted as having a generally round cross-sectional shape, it can be appreciated that the sheath body 26 can include other cross-sectional shapes or combinations of shapes, e.g., oval, rectangular, triangular, polygonal, and the like.

The delivery catheter 12 may include one or more, or a plurality of regions along its length having different configurations and/or characteristics. For example, the distal segment 30 of the sheath body 26 may have an outer diameter less than the outer diameter of the proximal segment 28 of the sheath body 26 to reduce the profile of the distal segment 30 and facilitate navigation in tortuous vasculature. Furthermore, the distal segment 30 may be more flexible than the proximal segment 28. Generally, the proximal segment 28 may be formed from material that is stiffer than the distal segment 30 of the sheath body 26, so that the proximal segment 28 has sufficient pushability to advance through the patient’s vascular system, while the distal segment 30 may be formed of a more flexible material so that the distal segment 30 may remain flexible and track more easily over a guidewire to access remote locations in tortuous regions of the vasculature. The sheath body 26 may be composed of suitable polymeric materials, metals and/or alloys, such as polyethylene, stainless steel or other suitable biocompatible materials or combinations thereof. In some instances, the proximal segment 28 may include a reinforcement layer, such a braided layer or coiled layer to enhance the pushability of the sheath body 26. The sheath body 26 may include a transition region between the proximal segment 28 and the distal segment 30.

The delivery catheter 12 comprises a distal port 34 in communication with the inner lumen 32 of the delivery catheter 12 and from which the vaso-occlusive device 22 is deployed. The delivery catheter 12 further comprises a proximal adapter 36 affixed to the proximal segment 28 of the sheath body 26 using suitable means, e.g., adhesive, welding, etc. The proximal adapter 36 comprises a central bore 38 (shown in phantom) in communication with the lumen 32 of the delivery catheter 12. The central bore 38 terminates in a proximal port 40 for allowing loading of the vaso-occlusive assembly 14 into the delivery catheter 12.

The delivery catheter 12 further comprises one or more radiopaque marker bands 44 (in this case, two distal and proximal bands 44a, 44b) disposed on the distal segment 30 of the delivery catheter 12 proximate the distal port 34, which can be identified using medical imaging technology (e.g., fluoroscopy). The distal band 44a may be used to locate the distal tip of the delivery catheter 12 within the patient's vasculature system, while the proximal band 44b may be used to locate the delivery catheter 12 relative to the partially or fully deployed vaso-occlusive device 22, such that the delivery catheter 12 and delivery wire 20 may be longitudinally aligned to ensure that the electrolytically severable joint 24 is located just distal to the distal port 34 of the delivery catheter 12 in contact with bodily fluids in the vasculature of the patient to facilitate electrolytic detachment of the vaso-occlusive device 22 from the delivery wire 20, as will be discussed in further detail below. The radiopaque marker bands 44 may be composed of a suitable radiopaque material, e.g., gold, platinum, palladium, tantalum, tungsten alloy, polymer material loaded with a radiopaque filler, and the like.

In general, the vaso-occlusive device 22 has a delivery configuration when restrained within a delivery catheter 12 (FIG. 1) and has a deployed configuration that conforms to the interior shape of an aneurysmal sac (not shown) when deployed from the delivery catheter 12 (FIG. 2). The vaso-occlusive device 22 may be pre-biased to form a cylinder, a cone, or other desired shape. The vaso-occlusive device 22 may be soft and its overall shape easily deformed. In the illustrated embodiment, the vaso-occlusive device 22 is shown as a helical coil formed of a wire having a suitable diameter, e.g., 1-6 mils. The diameter of the vaso-occlusive device 22, when in the delivery configuration, may be, e.g., 8-30 mils. The vaso-occlusive device 22 may have any suitable length desirable and appropriate for the site to be occluded, e.g., 1-50 cm. In alternative embodiments, the vaso-occlusive device 22 may take the form of a structure other than a coil, e.g., a braid. The vaso-occlusive device 22 may optionally be covered or connected with fibrous materials tied to the outside of the coil or braid. The vaso-occlusive device 22 may be composed of a suitable biocompatible and radio-opaque material, such as platinum, gold, tungsten, iridium, or alloys thereof or other metals. In the illustrated embodiment, the vaso-occlusive device 22 has an end cap or tip that prevents punctures of the aneurysmal sac when delivered therein.

The delivery wire 20 may be a coil, wire, or the like (e.g., a conventional guidewire, torqueable cable tube, or a hypotube), having a sufficient columnar strength to permit pushing of the vaso-occlusive device 22 into the aneurysmal sac. The delivery wire 20 may have a suitable outer diameter, e.g., 10-30 mils, and a suitable length, e.g., 50-300 cm. The material used to construct the delivery wire 20 is chosen to impart varying flexibility and stiffness characteristics to different portions of the delivery wire 20. For example, the delivery wire 20 may be formed of different materials along its length, for example materials having different moduli of elasticity, resulting in a difference in flexibility.

In the illustrated embodiment, the delivery wire 20 generally comprises an electrically conductive core wire 46 and a sleeve 48 composed of an electrically insulative material, such as, e.g., polytetrafluoroethylene, polyurethane, polyethylene, polypropylene, or other suitable polymeric material. The core wire 46 has a proximal segment 50 that extends proximal from the proximal portion 30 of the delivery catheter 12 for manipulation by the physician, a distal segment 52 to which the vaso-occlusive device 22 is attached, and a medial segment 54 disposed between the proximal segment 50 and the distal segment 52. The proximal segment 50 of the core wire 46 distally tapers downward to the medial segment 54. The distal segment 52 of the core wire 46 extends from the medial segment 54 and distally tapers further downward to provide flexibility to the distal end of the delivery wire 20. The delivery wire 20 may comprise a coil (not shown) affixed around the distal segment 52 of the core wire 46 to provide some columnar strength to the distal end of the delivery wire 20, while not detrimentally affecting the flexibility of the tapered distal segment 52 of the core wire 46. The sleeve 48 is disposed over the distal segment 52 of the core wire 46, and as discussed in further detail, serves to electrically isolate the portion of the distal segment 52 of the core wire 46 and that is proximal to the electrolytically severable joint 24, as well as the coil of the delivery wire 20, from the blood in the vasculature of the patient. The delivery wire 20 further comprises a radiopaque marker band 56 disposed over the sleeve 48, which can be identified using medical imaging technology (e.g., fluoroscopy). The marker band 56 may be used to locate the delivery catheter 12 relative to the partially or fully deployed vaso-occlusive device 22 (by aligning it relative to the proximal marker 44b of the delivery catheter 12), such that the delivery catheter 12 and delivery wire 20 may be longitudinally aligned to ensure that the electrolytically severable joint 24 is located just distal to the distal port 34 of the delivery catheter 12 in contact with bodily fluids in the vasculature of the patient to facilitate electrolytic detachment of the vaso-occlusive device 22 from the delivery wire 20, as will be discussed in further detail below. The radiopaque marker band 56 may be composed of a suitable radiopaque material, e.g., gold, platinum, palladium, tantalum, tungsten alloy, polymer material loaded with a radiopaque filler, and the like.

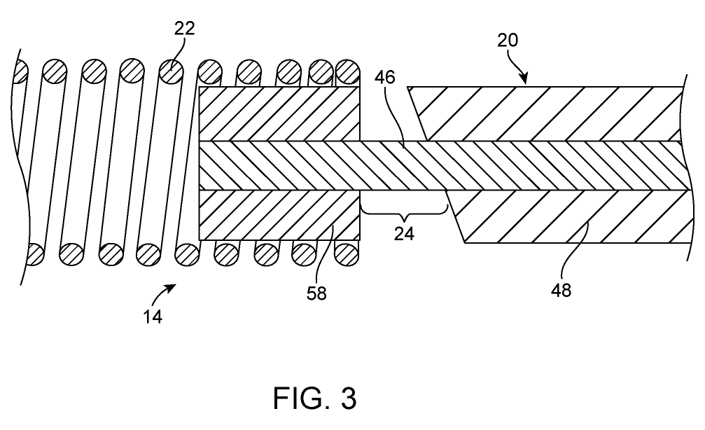

Referring further to FIG. 3, the vaso-occlusive device 22 is affixed to the distal portion 56 of the core wire 48 via an electrolytically-resistant bushing 58. The electrolytically severable joint 24 takes the form of an electrolytically degradable segment for electrolytically decoupling the vaso-occlusive device 22 from the delivery wire 20, and is located on the core wire 46 between the electrically insulative sleeve 48 and the vaso-occlusive device 22. Thus, when electrical current is supplied to the core wire 46, the electrical current flows to the electrolytically severable joint 24. However, the electrolytically severable joint 24 is not electrically insulated, and is, thus, more susceptible to electrolytic dissolution in blood than the portion of the core wire 46 covered with the electrically insulative sleeve 48 and the vaso-occlusive device 22. Thus, the electrolytically severable joint 24 will substantially or completely dissolve, allowing release of the vaso-occlusive device 22. Preferably, the length of the electrolytically severable joint 24 is not much greater than the diameter of the electrolytically severable joint 24. For example, the electrolytically joint 24 may be as short as 0.00085 inches, and typically no longer than 0.15 inches in length.

Referring back to FIGS. 1 and 2, the delivery wire 20 is bipolar in nature in that is capable of conducting electrical current to and from the electrolytically severable joint 24, and to this end, comprises a proximal terminal 66a (in this case, a power terminal) and a distal terminal 66b (in this case a return terminal) for respectively delivering electrical current from the electrolytic detachment device 16 to the electrolytically severable joint 24, and returning electrical current from the electrolytically severable joint 24 to the electrolytic detachment device 16. As will be described in further detail below, the delivery wire 20 is specifically constructed to provide electrically isolated forward and return paths to and from the electrolytically severable joint 24. In alternative embodiments, the delivery wire 20 may be monopolar in nature in that it is only capable of delivering electrical current from the electrolytic detachment device 16 to the electrolytically severable joint 24, which electrical current is then returned to a remote ground electrode in contact with the patient. In this case, the delivery wire 20 may comprise only a power terminal (not shown) for delivering electrical current from the electrolytic detachment device 16 to the electrolytically severable joint 24.

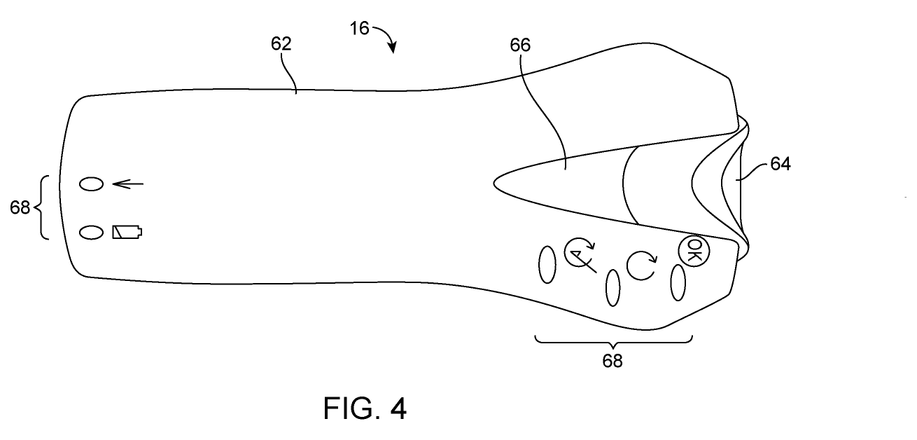

Referring further to FIG. 4, the electrolytic detachment device 16 may be operated by the physician to perform an electrolytic detachment procedure. The electrolytic detachment device 16 comprises an outer casing 62; an electrical port 64 (e.g., a funnel) configured for being electrically coupled to the delivery wire 20, and thus, the electrolytic severable joint 24, of the vaso-occlusive assembly 14, via the sterile adapter 18 (as will be described in further detail below), electronic componentry (not shown) contained within the outer casing 62 for delivering electrical current to electrolytic severable joint 24 of the vaso-occlusive assembly 14 in a controlled manner; an electrical current delivery button 66 affixed to the outer casing 62 for manually initiating the flow of electrical current from the electrolytic detachment device 16 to the electrolytic severable joint 24 of the vaso-occlusive assembly 14, and a plurality of indicators 68 configured for reporting different events to the physician during the electrolytic detachment procedure.

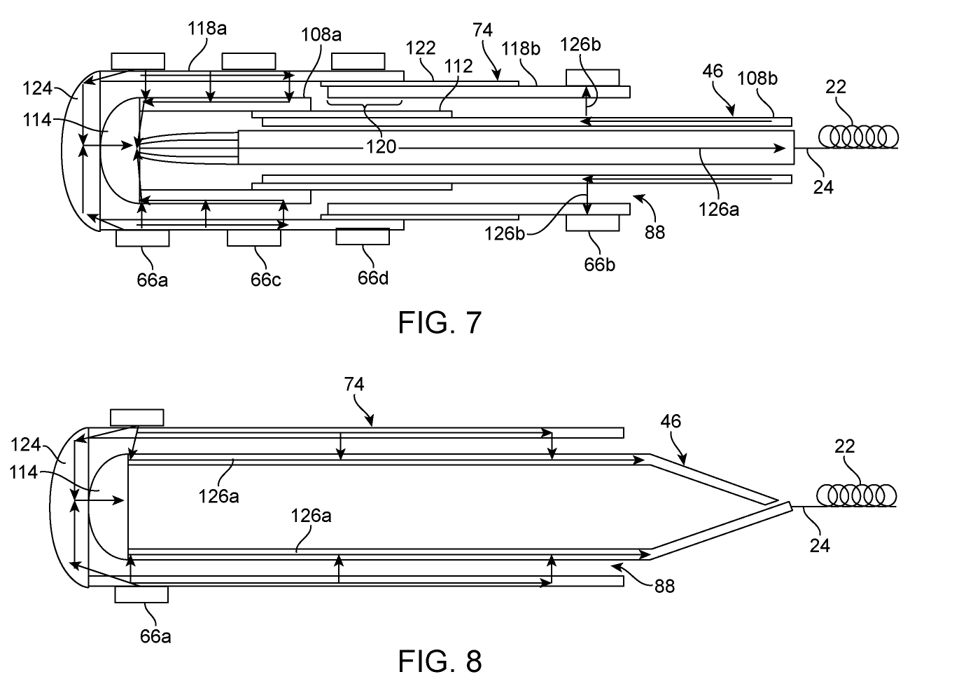

The outer casing 62 is composed of a suitable material, e.g., Acrylonitrile Butadiene Styrene (ABS) or polycarbonate, is shaped and sized to be ergonomically held by a physician with one hand. In the illustrated embodiment, the electrolytic detachment device 16 is bipolar in nature, in which case, the electrolytic detachment device 16 comprises both a power terminal 66a and a ground terminal 66b (shown in FIG. 7) contained within the electrical port 64 for connection to the electronic componentry contained in the outer case 62. In an alternative embodiment, the electrolytic detachment device 16 may comprise additional terminals (not shown) contained within the electrical port 64, so that, e.g., the electrolytic detachment device 16 may identify the type of vaso-occlusive assembly 22 electrically coupled to the electrolytic detachment device 16. In this manner, the electrolytic detachment device 16 may deliver electrical current to the vaso-occlusive assembly 22 in accordance with electrical parameters corresponding to the type of vaso-occlusive assembly 22 identified by the electrolytic detachment device 16. In an alternative embodiment, the electrolytic detachment device 16 is monopolar in nature, in which case, the ground terminal will not be contained in, but rather will be remote from, the electrical port 64 (e.g., in the outer case 62 opposite to the electrical port 64) for connection to a cable associated with a ground electrode (not shown) placed in contact with the patient. The sterile adapter 18 may comprise an additional hub assembly (not shown) affixed to the sterile sleeve 70 to facilitate electrical connection between the separate ground terminal of the electrolytic detachment device 16 and a cable connector associated with the ground electrode.

The electronic componentry is configured for delivering electrical current to the electrolytic severable joint 24 of the vaso-occlusive assembly 14 during one or more electrolytic detachment cycles until the vaso-occlusive device 22 electrolytically detaches from the delivery wire 20, as well as reporting to the physician of various events that occur during the electrolytic detachment procedure. In the illustrated embodiment, the electrical current delivery button 66 takes the form of a push button, which can be depressed to manually command the electrolytic detachment device 16 to perform an electrolytic detachment cycle (i.e., a time period during which electrical current is delivered from the electronic componentry to the electrolytic severable joint 24 of the vaso-occlusive assembly 14). Alternatively, other types of manual controls (e.g., a switch, a dial, etc.) may be used to manually command the electrolytic detachment device 16 to perform an electrolytic detachment cycle. The indicators 68 are configured for reporting different events to the physician during the electrolytic detachment procedure, e.g., the powering on of the electrolytic detachment device 16, active delivery of the electrical current from the electrolytic detachment device 16 to the vaso-occlusive assembly 22, an assessment that a successful electrolytic detachment event has occurred, indication that the battery is low, etc.

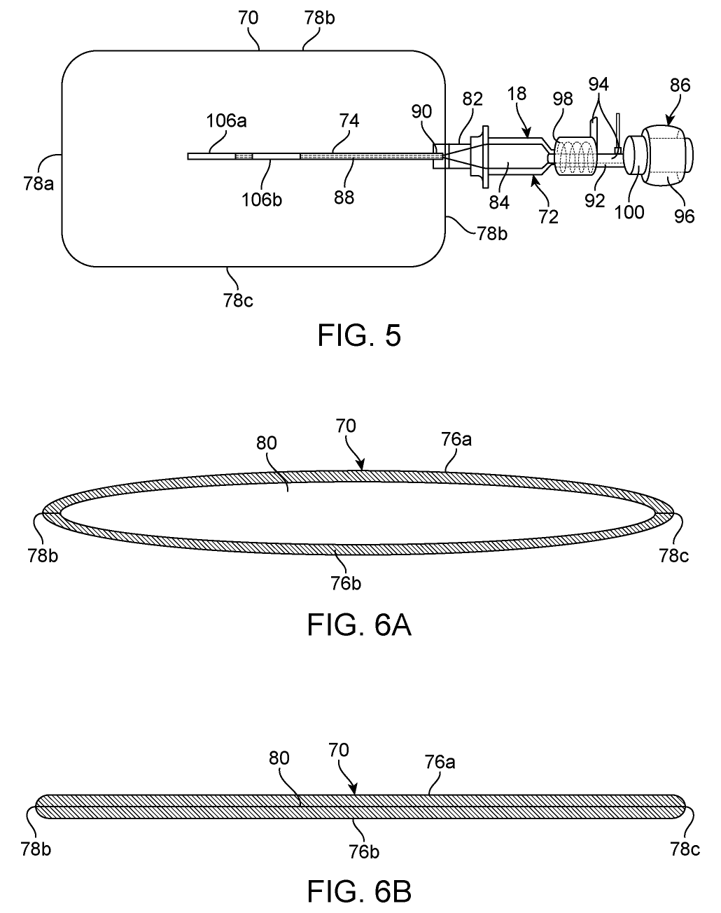

Referring further to FIGS. 5, 6A, and 6B, the sterile adapter 18 is designed to be disposable, and as such, is comprised of components that are inexpensive compared to the electrolytic detachment device 16. The sterile adapter 18 generally comprises a sterile sleeve 70 sized to removably contain the electrolytic detachment device 16; a hub assembly 72 affixed to the sterile sleeve 70 for receiving a proximal end of the core wire 46 of the vaso-occlusive assembly 14; and an electrically conductive shaft 74 affixed to the hub assembly 72 and contained within the sterile sleeve 70.

In the illustrated embodiment, the size and shape of the handheld instrument designed to be contained in the sterile sleeve 70, and in this case the electrolytic detachment device 16, will dictate the size and shape of the sterile sleeve 70. In the illustrated embodiment, the sterile sleeve 70 takes the form of a pouch or bag composed of two planar rectangular sheets 76a, 76b (shown best in FIGS. 6A and 6B) that are joined together at three edges (in this case, the distal edge 78a, and the opposing longitudinal edges 78b, 78c). The sterile sleeve 70 is preferably closable, sealable, and openable. In the illustrated embodiment, the sterile sleeve 70 has a sealable opening 80 located at a proximal edge 78d through which the electrolytic detachment device 16 may be introduced into the interior of the sterile sleeve 70. Although the sterile sleeve 70 is illustrated as being rectangular in shape, it should be appreciated that the sterile sleeve 70 may have any shape, including elliptical, hexagonal, ovular, triangular, etc., or any other shape that can accommodate insertion and removal of the electrolytic detachment device 16.

In the illustrated embodiment, the opening 80 is reversibly sealable, such that, prior to sealing the opening 80, the electrolytic detachment device 16 may be introduced through the opening 80 into the interior of the sterile sleeve 70, and such opening 80 can then be unsealed, such that the electrolytic detachment device 16 may be removed from the interior of the sterile sleeve 70 through the unsealed opening 80 without destroying the sterile sleeve 70. For example, the opening 80 may take the form of a zipper-type or slidable channel-type mechanism that can be pressed between the thumb and the forefinger and slid along the proximal edge 78d of the sterile sleeve 70 in one direction or otherwise zipping the opening 80 to seal the electrolytic detachment device 16 within the sterile sleeve 70. The sealed opening 80 can then be reopened by pulling the planar rectangular sheets 76a, 76b apart at the proximal edge 78d or otherwise unzipping the sealed opening 80. In alternative embodiments, the opening 80 may be permanently sealable, e.g., with a plastic sealer, such that, prior to permanently sealing the opening 80, the electrolytic detachment device 16 may be introduced through the opening 80 into the interior of the sterile sleeve 70, and then after the opening 80 is permanently sealed, the electrolytic detachment device 16 may be removed from the interior of the sterile sleeve 70 by ripping or otherwise cutting a hole in the sterile sleeve 70. The sterile sleeve 70 is composed of a flexible polymer material, such as, e.g., polyethylene or polypropylene. Preferably, the material from which the sterile sleeve 70 is composed is optically transparent, such that the electrical current delivery button 66 and indicators 68 (shown in FIG. 4) can be visualized while the electrolytic detachment device 16 is contained within the sterile sleeve 70.

The hub assembly 72 comprises a hub 82 affixed to the sterile sleeve 70, such that the proximal end of the core wire 46 of the vaso-occlusive assembly 14 may be introduced from the exterior of the sterile sleeve 70 into the electrically conductive shaft 74 (described in further detail below) via the hub 82. The hub 82 comprises a funnel-shaped port 84 through which the proximal end of the core wire 46 may be passed into the electrically conductive shaft 74, as will be described in further detail below. In the illustrated embodiment, the hub 82 is disposed through the distal edge 78a of the sterile sleeve 70, such that a distal portion of the hub 82 resides outside of the sterile sleeve 70, while a proximal portion of the hub 82 resides inside of the sterile sleeve 70. During the manufacture of the sterile adapter 18, the two planar rectangular sheets 76a, 76b of the sterile sleeve 70 may be joined together over the hub 82 at the distal edge 78a, such that that interior of the sterile sleeve 70 (external to the electrically conductive shaft 74) is sealed from the exterior of the sterile sleeve 70 when the opening 80 of the sterile sleeve 70 is sealed. The hub 82 may be composed of a suitably rigid material, such as, e.g., a rigid polymer (e.g., polystyrene, nylon, polycarbonate, or methacrylate).

The electrically conductive shaft 74 comprises a lumen 88 into which the proximal end of the core wire 46 of the vaso-occlusive assembly 14 may be slidably disposed when received by the hub 82. To this end, the distal end of the electrically conductive shaft 74 is affixed to the hub 82, such that the lumen 88 of the electrically conductive shaft 74 is coupled to the port 84 of the hub 82. For example, the hub 82 may comprise a proximal lumen 90 that couples with the port 84 and in which the distal end of the electrically conductive shaft 74 is disposed and suitably affixed therein, e.g., via bonding.

The hub assembly 72 optionally comprises a rotating hemostasis valve (RHV) 86 to which the hub 82 is mateable. The RHV 86 comprises a cylindrical tube bhaving a central lumen 94, a conventional male Touhy-Borst connector 96 rotatably affixed to the distal end of the cylindrical tube 92, and a Luer connector 98 affixed to the proximal end of the cylindrical tube 92. The Luer connector 98 may be mated to the hub 82 of the hub assembly 72. For example, the Luer connector 98 may take the form of complementary Luer connector that mates with the Luer connector 98. The proximal end of the core wire 46 of the vaso-occlusive assembly 14 may then be inserted through the central lumen 94 of the RHV 86, through the port 84 of the hub 82, and into the lumen 88 of the electrically conductive shaft 74. The RHV 86 may further comprise one or more seals (not shown) disposed within the central lumen 94 for sealing fluid flow between the outer surface of the core wire 46 and the inner surface of the cylindrical tube 92. The RHV 86 is configured for reversibly affixing the core wire 46 relative to the hub 82. To this end, the RHV 86 further comprises a compression nut 100 configured compressing one of the seals against the outer surface of the core wire 46 to affix the core wire 46 when the Touhy-Borst connector 96 is rotated in one direction, and decompressing the seal(s) away from the outer surface of the core wire 46 to release the core wire 46 when the Touhy-Borst connector 96 is rotated in the opposite direction.

The proximal end of the electrically conductive shaft 74 is configured for being snugly inserted into the electrical port 64 of the electrolytic detachment device 16. In the illustrated embodiment, the sterile adapter 18 is bipolar in nature in that it is capable of delivering electrical current from the electrolytic detachment device 16 to the electrolytically severable joint 24 of the vaso-occlusive assembly 14, and returning electrical current from the electrolytically severable joint 24 of the vaso-occlusive assembly 14 to the electrolytic detachment device 16. To this end, the sterile adapter 18 comprises a proximal terminal 106a (in this case, a power terminal) and a distal terminal 106b (in this case a distal terminal) for respectively delivering electrical current from the electrolytic detachment device 16 to the electrolytically severable joint 24, and returning electrical current from the electrolytically severable joint 24 to the electrolytic detachment device 16. As will be described in further detail below, the electrically conductive shaft 74 is specifically constructed to provide electrically isolated forward and return paths to and from the electrolytically severable joint 24. In alternative embodiments, the sterile adapter 18 may be monopolar in nature in that it is only capable of delivering electrical current from the electrolytic detachment device 16 to the electrolytically severable joint 24, which electrical current is then returned to a remote ground electrode in contact with the patient. In this case, the delivery wire 20 may comprise only a power terminal (not shown) for delivering electrical current from the electrolytic detachment device 16 to the electrolytically severable joint 24.

Significantly, when the proximal end of the core wire 46 of the vaso-occlusive assembly 14 is fully inserted into the lumen 88 of the electrically conductive shaft 74 via the port 84 of the hub 82 of the sterile adapter 18, the power and ground terminals 66a, 66b of the vaso-occlusive assembly 14 (shown in FIGS. 1-2) respectively electrically contact the power and ground terminals 106a, 106b of the sterile adapter 18, and when the proximal end of the electrically conductive shaft 74 is fully inserted into the electrical port 64 of the electrolytic detachment device 16 (shown in FIG. 5), the power and ground terminals 106a, 106b of the sterile adapter 18 respectively electrically contact the power and ground terminals 66a, 66b of the electrolytic detachment device 16.

As a result, while the electrolytic detachment device 16 is contained and sealed within the sterile sleeve 70 of the sterile adapter 18, the power and ground terminals 66a, 66b of the vaso-occlusive assembly 14 are respectively electrically coupled of the power and ground terminals 66a, 66b of the electrolytic detachment device 16 respectively via the power and ground terminals 106a, 106b of the sterile adapter 18, such that, in response to manual actuation of the electrical current delivery button 66 of the electrolytic detachment device 16, electrical current may be delivered from the electrolytic detachment device 16 to the electrolytically severable joint 24 of the vaso-occlusive assembly 14, and electrical current may be returned from the electrolytically severable joint 24 of the vaso-occlusive assembly 14 to the electrolytic detachment device 16, while physically isolating the electrolytic detachment device 16 from the vaso-occlusive assembly 14. The proximal end of the electrically conductive shaft 74 is sealed, such that the electrical port 64 of the electrolytic detachment device 16, while contained and sealed within the sterile sleeve 70, such that contaminates cannot ingress through the port 84 of the hub 82, through the lumen 88 of the electrically conductive shaft 74, and into the electrical port 64 of the electrolytic detachment device 16.

Of course, if the vaso-occlusive assembly 14, sterile adapter 18, electrolytic detachment device 16 are monopolar in nature, while the electrolytic detachment device 16 is contained and sealed within the sterile sleeve 70 of the sterile adapter 18, only the power terminal of the vaso-occlusive assembly 14 will be electrically coupled of only the power terminal of the electrolytic detachment device 16 via the only the power terminal of the sterile adapter 18, such that, in response to manual actuation of the electrical current delivery button 66 of the electrolytic detachment device 16, electrical current may be delivered from the electrolytic detachment device 16 to the electrolytically severable joint 24 of the vaso-occlusive assembly 14, and electrical current may be returned from the electrolytically severable joint 24 of the vaso-occlusive assembly 14 to a separate ground electrode placed into contact with the patient remote from the electrolytic detachment device 16, while physically isolating the electrolytic detachment device 16 from the vaso-occlusive assembly 14.

Notably, the spacing and sizes of the power and ground terminals 66a, 66b of the vaso-occlusive assembly 14 and corresponding power and ground terminals 106a, 106b of the sterile adapter 18 may be selected, such that one or more status terminals 66c, 66d (shown in FIG. 7) in the electrical port 64 of the electrolytic detachment device 16 may electrically contact one or both of the power and ground terminals 106a, 106b of the sterile adapter 18 in a unique manner in order to determine the type of vaso-occlusive assembly 14 in current use with the electrolytic detachment device 16, as will be described in further detail below.

Referring now to FIG. 7, one specific implementation of the core wire 46 of the vaso-occlusive assembly 14 and electrically conductive shaft 74 of the sterile adapter 18 will be described. In this implementation, the vaso-occlusive assembly 14 and sterile adapter 18 are bipolar in nature.

The core wire 46 of the vaso-occlusive assembly 14 comprises a proximal electrically conductive shaft portion (in this case, a hypotube) 108a, a distal electrically conductive shaft portion (in this case, a hypotube) 108b partially overlapping over the proximal hypotube 108a to create an overlapping region 110, and an electrically insulative element (in this case, a layer) 112 disposed between, and spanning the overlapping region 110 of, the proximal and distal hypotubes 108a, 108b. The proximal and distal hypotubes 108a, 108b may be composed of a suitable electrically conductive material, such as, e.g., stainless steel, whereas the electrically insulative layer 112 may be composed of a suitable electrically insulative material, such as, e.g., polytetrafluoroethylene, polyurethane, polyethylene, polypropylene, or other suitable polymeric material. Notably, the exposed portion of the proximal hypotube 108a corresponds to the power terminal 66a (illustrated in FIGS. 1-2), whereas the exposed portion of the distal hypotube 108b corresponds to the ground terminal 66b (illustrated in FIGS. 1-2). The core wire 46 further comprises an electrically conductive epoxy dome 114 formed on the free end of the proximal hypotube 108a, and an insulated conductor 116 affixed between the electrically conductive epoxy dome 114 and the electrolytically severable joint 24.

Similarly, the electrically conductive shaft 74 of the sterile adapter 18 comprises a proximal electrically conductive shaft portion (in this case, a hypotube) 118a, a distal electrically conductive shaft portion (in this case, a hypotube) 118b partially overlapping over the proximal hypotube 118a to create an overlapping region 120, and an electrically insulative element (in this case, a layer) 122 disposed between, and spanning the overlapping region 120 of, the proximal and distal hypotubes 118a, 118b. The proximal and distal hypotubes 118a, 118b may be composed of a suitable electrically conductive material, such as, e.g., stainless steel, whereas the electrically insulative layer 122 may be composed of a suitable electrically insulative material, such as, e.g., polytetrafluoroethylene, polyurethane, polyethylene, polypropylene, or other suitable polymeric material. Notably, the exposed portion of the proximal hypotube 118a corresponds to the power terminal 106a (illustrated in FIG. 5), whereas the exposed portion of the distal hypotube 118b corresponds to the ground terminal 106b (illustrated in FIG. 5). The electrically conductive shaft 74 further comprises an electrically conductive epoxy dome 124 formed on the free end of the proximal hypotube 118a, thereby sealing the lumen 88 of the electrically conductive shaft 74, as discussed above.

When the proximal end of the core wire 46 of the vaso-occlusive assembly 14 is fully inserted into the lumen 88 of the electrically conductive shaft 74 of the sterile adapter 18, as illustrated in FIG. 7, the electrically conductive epoxy domes 114, 124 are in electrical contact with each other, the proximal hypotubes (power terminals 66a, 106a) 108a, 118a are in electrically contact with each other, and the distal hypotubes 108b, 118b (ground terminals 66b, 106b) are in electrical contact with each other. Furthermore, when the proximal end of the electrically conductive shaft 74 of the sterile adapter 18 is fully inserted into the electrical port 64 of the electrolytic detachment device 16, as illustrated in FIG. 7, the proximal and distal hypotubes 108a, 108b (power and ground terminals 106a, 106b) of the electrically conductive shaft 74 are respectively in electrical contact with the power and ground terminals 66a, 66b of the electrolytic detachment device 16.

Thus, a forward electrical path 126a is created from the power terminal 66a of the electrolytic detachment device 16, through the proximal hypotube 118a/electrically conductive epoxy dome 124 of the electrically conductive shaft 74 of the sterile adapter 18, through the proximal hypotube 108a/electrically conductive dome 114, and along the insulated conductor 116 to the electrolytically severable joint 24, of the vaso-occlusive assembly 14, while a return electrical path 126b is created from the electrolytically severable joint 24, along the distal hypotube 108b of the vaso-occlusive assembly 14, through the distal hypotube 118b of the sterile adapter 18, and to the ground terminal 66b of the electrolytic detachment device 16.

Notably, the proximal tube 118a of the sterile adapter is in electrical contact with one or more of the status terminals 66c, 66d of the electrolytic detachment device 16. The type of the vaso-occlusive assembly 14 to be used with the electrolytic detachment device 16 may be encoded into the length of the proximal tube 118a of the electrically conductive shaft 74 of the sterile adapter 18, such that the electrolytic detachment device 16 may identify the type of vaso-occlusive assembly 14 to which it is coupled. For example, in the illustrated embodiment, the length of the proximal tube 118a has been selected, such that it spans and is in electrical contact with all three of the power terminal 66a and status terminals 66c-66d, thereby electrically shorting these three terminals 66a-66c together, and indicating to the electrolytic detachment device 16 that the vaso-occlusive assembly 14 is of a first type. The length of the proximal tube 118a may be made shorter, such that it spans and is in electrical contact with the power terminal 66a and only the status terminal 66b, thereby only electrically shorting these two terminals 66a-66b (while the status terminal 66c remains open), and indicating to the electrolytic detachment device 16 that the vaso-occlusive assembly 14 is of a second type.

In the alternative embodiment where the vaso-occlusive assembly 14 and sterile adapter 18 are monopolar in nature, the core wire 46 of the vaso-occlusive assembly 14 simply comprises a single shaft (or hypotube) electrically to the electrolytically severable joint 24 and to which the electrically conductive epoxy dome 114 is affixed, whereas the electrically conductive shaft 74 of the sterile adapter 18 comprises a single electrically conductive shaft to which the electrically conductive epoxy dome 124 is affixed, as illustrated in FIG. 8. In this case, when the proximal end of the core wire 46 of the vaso-occlusive assembly 14 is fully inserted into the lumen 88 of the electrically conductive shaft 74 of the sterile adapter 18, the electrically conductive epoxy domes 114, 124 are in electrical contact with each other, and the core wire 46 and electrically conductive shaft 74 are in electrical contact with each other. Furthermore, when the proximal end of the electrically conductive shaft 74 of the sterile adapter 18 is fully inserted into the electrical port 64 of the electrolytic detachment device 16, the electrically conductive shaft 74 is in electrical contact with the power terminal 66a of the electrolytic detachment device 16. Thus, a forward electrical path 126a is created from the power terminal 66a of the electrolytic detachment device 16, through the electrically conductive epoxy dome and electrically conductive shaft 74 of the sterile adapter 18, through the electrically conductive dome 114 and along the core wire 48 to the electrolytically severable joint 24, of the vaso-occlusive assembly 14. The sterile adapter 18 may comprise an additional hub assembly (not shown) for insertion into a separate ground port (not shown) of the electrolytic detachment device 16. Such additional hub assembly may comprise a cable assembly (not shown) having a plug residing within the sterile sleeve 70 for connection to a separate ground port of the electrolytic detachment device 16, a cable extending from the plug through the sterile sleeve 70, and another plug for connection to a ground electrode.

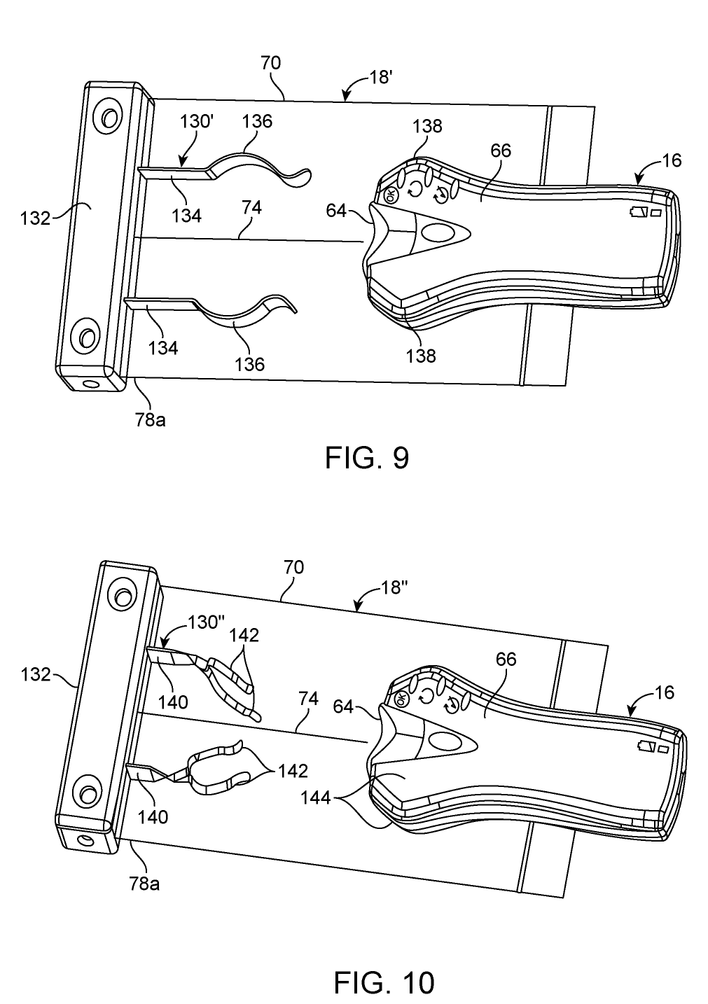

In optional embodiments illustrated in FIGS. 9 and 10, a sterile adapter 18’ further comprises a clamp mechanism 130 (clamp mechanism 130’ illustrated in FIG. 9 or clamp mechanism 130” illustrated in FIG. 10) configured for grasping the electrolytic detachment device 16 when contained within the sterile sleeve 70 of the sterile adapter 18. Each of the clamp mechanisms 130’, 130” comprises a block 132 affixed to the distal edge 78a of the sterile sleeve. Each of the clamp mechanism 130’, 130” comprises clamp arms that are interior to the sterile sleeve 70 and have their bases affixed to the block 132.

In particular, the clamp mechanism 130’ illustrated in FIG. 9 comprises a pair of resilient arms 134, each having an outwardly curved region 136 that are spaced from each other in a manner, such that the outwardly curved regions 136 of the respective resilient arms 134 grasp opposing curved edges 138 of the outer casing 62 of the electrolytic detachment device 16. Thus, when the electrolytic detachment device 16 is inserted through the opening 80 and into the interior of the sterile sleeve 70, the electrically conductive shaft 74 of the sterile adapter 18 may be inserted into the electrical port 64 of the electrolytic detachment device 16 while opposing curved edges 138 of the outer casing 62 of the electrolytic detachment device 16 urge the pair of resilient arms 134 away from each other, until the outwardly curved regions 136 of the respective resilient arms 134 coincide with and laterally grasp the opposing curved edges 138 of the outer casing 62 of the electrolytic detachment device 16.

In contrast, the clamp mechanism 130” illustrated in FIG. 10 comprises a pair of arms 140, each of which comprises a pair of resilient fingers 142 that are spaced from each other in a manner, such that pairs of resilient fingers 142 grasp opposing top and bottom surfaces 144 of the outer case 62 of the electrolytic detachment device 16. Thus, when the electrolytic detachment device 16 is inserted through the opening 80 and into the interior of the sterile sleeve 70, the electrically conductive shaft 74 of the sterile adapter 18 may be inserted into the electrical port 64 of the electrolytic detachment device 16 while opposing curved edges 138 of the outer casing 62 of the electrolytic detachment device 16 urge the pairs of resilient fingers 142 away from each other while they laterally grasp the opposing top and bottom surfaces 144 of the outer casing 62 of the electrolytic detachment device 16.

In this manner, movement between the proximal end of the electrically conductive shaft 74 of the sterile adapter 18 and the electrical port 64 of the electrolytic detachment device 16 may be prevented, thereby ensuring that that the electrically conductive shaft 74 remains in electrical contact with the power terminal 66a and/or ground terminal 66b of the electrolytic detachment device 16.

Although particular embodiments have been shown and described herein, it will be understood by those skilled in the art that they are not intended to limit the disclosed inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

What is claimed is:

1. A sterile adapter for electrically coupling an electrically conductive wire of a medical device to an electrical port of a handheld instrument, comprising:
a sterile sleeve sized to removably contain the handheld instrument;
a hub assembly affixed to the sterile sleeve for receiving a proximal end of the electrically conductive wire of the medical device; and
an electrically conductive shaft affixed to the hub assembly and contained within the sterile sleeve, the electrically conductive shaft having a distal end affixed to the hub assembly and a proximal end configured for being inserted into the electrical port of the handheld instrument, the electrically conductive shaft having a lumen into which the proximal end of the electrically conductive wire of the medical device may be slidably disposed when received by the hub assembly.

2. The sterile adapter of claim 1, wherein the sterile sleeve is optically transparent.

3. The sterile adapter of claim 1, wherein the sterile sleeve has a sealable opening into which the handheld instrument, and from which the handheld instrument may be removed.

4. The sterile adapter of claim 1, further comprising a clamping mechanism affixed to the sterile sleeve, the clamping mechanism configured for grasping the handheld instrument when contained within the sterile sleeve.

5. The sterile adapter of claim 1, wherein the hub assembly comprises a hub affixed to the sterile sleeve, and a rotating hemostasis valve (RHV) to which the hub is affixed, for reversibly affixing the electrically conductive wire of the medical device relative to the hub.

6. The sterile adapter of claim 1, wherein the electrically conductive shaft is a hypotube.

7. The sterile adapter of claim 1, wherein the lumen of the electrically conductive shaft is closed at the proximal end of the electrically conductive shaft.

8. The sterile adapter of claim 7, wherein the proximal end of the electrically conductive shaft has an electrically conductive epoxy cap.

9. The sterile adapter of claim 1, wherein the electrically conductive shaft has a proximal shaft portion, a distal shaft portion, and an electrically insulative element disposed between the proximal and distal shaft portions.

10. The sterile adapter of claim 9, wherein the proximal shaft portion is a proximal hypotube, the second shaft portion is a distal hypotube, wherein one of the proximal and distal hypotubes is partially inserted into the other of the proximal and distal hypotubes to create an overlapping region, and wherein the electrically insulative element is an electrically insulative layer disposed between, and spanning the overlapping region of the proximal and distal hypotubes.

11. A medical kit, comprising:
a medical device comprising an electrically conductive wire;
a handheld instrument comprising an electrical port having at least one electrical terminal; and
a sterile adapter comprising a sterile sleeve sized to removably contain the handheld instrument, a hub assembly affixed to the sterile sleeve for receiving a proximal end of the electrically conductive wire of the medical device, and an electrically conductive shaft affixed to the hub assembly and contained within the sterile sleeve, the electrically conductive shaft having a distal end affixed to the hub assembly and a proximal end configured for being inserted into the electrical port of the handheld instrument, such that the at least one electrical terminal of the handheld instrument electrically contacts the electrically conductive shaft, the electrically conductive shaft having a lumen into which the proximal end of the electrically conductive wire of the medical device may be slidably disposed when received by the hub assembly, such that electrically conductive wire is electrically coupled to the at least one electrical terminal of the handheld instrument via the electrically conductive shaft of the sterile adapter.

12. The medical kit of claim 11, wherein the medical device is a vaso-occlusive assembly that comprises a vaso-occlusive device electrolytically attached to a distal end of the electrically conductive wire, and wherein the handheld instrument is a handheld electrolytic detachment device.

13. The medical kit of claim 11, wherein the handheld instrument comprises at least one control for delivering electrical current to the medical device via the at least one electrical terminal, and wherein the sterile sleeve is optically transparent.

14. The medical kit of claim 11, wherein the sterile sleeve has a sealable opening into which the handheld instrument may be inserted, and from which the handheld instrument may be removed.

15. The medical kit of claim 11, wherein the sterile adapter further comprises a clamping mechanism affixed to the sterile sleeve, the clamping mechanism configured for grasping the handheld instrument when contained within the sterile sleeve.

16. The medical kit of claim 11, wherein the hub assembly comprises a hub affixed to the sterile sleeve, and a rotating hemostasis valve (RHV) to which the hub is affixed, for reversibly affixing the electrically conductive wire of the medical device relative to the hub.

17. The medical kit of claim 11, wherein the electrically conductive shaft is a hypotube.

18. The medical kit of claim 11, wherein the lumen of the electrically conductive shaft is closed at the proximal end of the electrically conductive shaft.

19. The medical kit of claim 18, wherein the proximal end of the electrically conductive shaft has an electrically conductive epoxy cap.

20. The medical kit of claim 11, wherein the at least one electrical terminal of the handheld instrument comprises a power terminal disposed in the electrical port, wherein the medical device comprises a power terminal disposed on the electrically conductive wire, and wherein the sterile adapter comprises a power terminal disposed on the electrically conductive shaft, such that power terminal of the medical device electrically contacts the power terminal of the sterile adapter when the proximal end of the electrically conductive wire is inserted into the lumen of the electrically conductive shaft of sterile adapter, and such that the power terminal of the handheld instrument electrically contacts the power terminal of the sterile adapter when the proximal end of the electrically conductive shaft of the sterile adapter is inserted into the electrical port of the handheld instrument.

21. The medical kit of claim 20, wherein the at least one electrical terminal of the handheld instrument further comprises a ground terminal, wherein the medical device further comprises a ground terminal disposed on the electrically conductive wire, and wherein the sterile adapter further comprises a ground terminal disposed on the electrically conductive shaft, such that ground terminal of the medical device electrically contacts the ground terminal of the sterile adapter when the proximal end of the electrically conductive wire is inserted into the lumen of the electrically conductive shaft of sterile adapter, and such that the ground terminal of the handheld instrument electrically contacts the ground terminal of the sterile adapter when the proximal end of the electrically conductive shaft of the sterile adapter is inserted into the electrical port of the handheld instrument.

22. The medical kit of claim 21,
wherein the electrically conductive wire of the vaso-occlusive assembly comprises a first proximal shaft portion, a first distal shaft portion, and a first electrically insulative element disposed between the first proximal shaft portion and the first distal shaft portion, thereby forming the power terminal and the ground terminal of the vaso-occlusive assembly; and
wherein the electrically conductive shaft of the sterile adapter comprises a second proximal shaft portion, a second distal shaft portion, and a second electrically insulative element disposed between the second proximal shaft portion and the second distal shaft portions, thereby forming the power terminal and the ground terminal of the sterile adapter.

23. The medical kit of claim 22,
wherein the first proximal shaft portion is a first proximal hypotube, the first distal shaft portion is a first distal hypotube, wherein one of the first proximal hypotube and the first distal hypotube is partially inserted into the other of the first proximal hypotube and the first distal hypotube to create an overlapping region, and wherein the first electrically insulative element is a first electrically insulative layer disposed between, and spanning the overlapping region, of the first proximal hypotube and the first distal hypotube; and
wherein the second proximal shaft portion is a second proximal hypotube, the second distal shaft portion is a second distal hypotube, wherein one of the second proximal hypotube and the second distal hypotube is partially inserted into the other of the second proximal hypotube and the second distal hypotube to create an overlapping region, and wherein the second electrically insulative element is a second electrically insulative layer disposed between, and spanning the overlapping region, of the second proximal hypotube and the second distal hypotube.
